(19) United States
(12) Patent Application Publication
(10) Pub. No.: US 2009/0246273 A1
(43) Pub. Date: Oct. 1, 2009

(54) Ketorolac Sublingual Spray for the Treatment of Pain

(76) Inventor: Abeer M. Al-Ghananeem, Lexington, KY (US)

Correspondence Address: FROST BROWN TODD, LLC, 2200 PNC CENTER, 201 E. FIFTH STREET, CINCINNATI, OH, 45202, US

(21) Appl. No.: 12/410,928

(22) Filed: Mar. 25, 2009

Related U.S. Application Data
(60) Provisional application No. 61/039,986, filed on Mar. 27, 2008.

(51) Int. Cl. A61K 9/127; A61K 9/14; A61K 9/12; A61K 31/407

(52) U.S. Cl. 424/450; 424/489; 424/45; 514/413

(57) ABSTRACT

The present invention provides for compositions and methods for accelerating the rate of delivery of ketorolac to the systemic circulation by sublingual spray administration under the tongue to provide a rapid response in the treatment of pain, especially acute pain associated with postoperative pain and migraine headache. Compositions of ketorolac formulated for sublingual delivery as liquid spray are provided. Also provided are methods of treatment and management of pain.

### CROSS-REFERENCE TO RELATED APPLICATIONS

This application claims priority to U.S. Provisional Patent Application Ser. No. 61/039,986, which was filed on Mar. 27, 2008, the entirety of which is incorporated herein by reference for all purposes.

### FIELD OF THE INVENTION

The present invention relates to methods for delivering active agents comprise ketorolac free acid, ketorolac tromethamine, or any pharmaceutically acceptable salts through sublingual administration.

More specifically, the invention relates to a ketorolac liquid spray formulation for sublingual administration, used in the treatment and management of pain, especially pain associated with postoperative pain and migraine headache.

### BACKGROUND OF THE INVENTION

Several recent studies have examined the use of ketorolac in the emergency treatment of migraine headache and postoperative pain. Migraine treatment by administering drugs via the oral route is, at best, incomplete, abortive, and somewhat ineffective. Stomach upset is the most common side effect of ketorolac. Vomiting, bloating, gas, loss of appetite, dizziness, drowsiness, blurred vision, may also occur. This is mainly due to the dysfunction of the gastrointestinal tract, the dilation of the stomach, and the closure of the pyloric sphincter during a migraine attack. Moreover, the need for repeated injections due to the rapid metabolism of ketorolac (half-life 4-5 hr) makes the intravenous mode of delivery inconvenient in many situations. Thus, a strong need exists for alternative modes of ketorolac delivery that do not have gastrointestinal side effects and may be more convenient than injections. The principal objective of this invention is to develop an effective sublingual spray formulation comprising ketorolac free acid, ketorolac tromethamine, or any pharmaceutically acceptable, that exhibited enhanced bioavailability and onset of action.

### BRIEF SUMMARY OF THE INVENTION

The present invention relates to compositions comprising ketorolac for sublingual delivery in liquid spray formulations. The invention is also directed to methods of treatment comprising administering ketorolac as liquid spray formulation to the sublingual tissue under the tongue. The inventive methods may improve bioavailability relative to oral dosage forms, especially in those patients with abnormally slow gastric emptying. The methods may provide treatment for a variety of conditions such as in the emergency treatment of migraine headache and postoperative pain that are amenable to amelioration by ketorolac administration, without the occurrence of possible side effects associated with oral ingestion.

The present invention also provides for pharmaceutical compositions comprising ketorolac free acid, ketorolac tromethamine, or a pharmaceutically acceptable salt thereof and a pharmaceutically acceptable carrier, wherein the ketorolac or pharmaceutically acceptable salt thereof is provided in a form suitable for sublingual administration.

The present invention provides a ketorolac spray for sublingual administration, to be used in the treatment and amelioration of pain, as well as the treatment and amelioration of similar symptoms. The present invention also provides methods of treatment as well as a metered dosage system for use in administration of the ketorolac spray.

In one of its embodiments, a pharmaceutical composition comprising ketorolac or a pharmaceutically acceptable salt thereof and a pharmaceutically acceptable carrier, wherein the ketorolac or pharmaceutically acceptable salt thereof is provided in a form suitable for sublingual spray administration is provided.

In another embodiment, a liquid spray formulation, comprising (i) ketorolac or pharmaceutically acceptable salt or free acid thereof, (ii) buffered water; and (iii) a polar organic solvent is provided. The polar organic solvent is present in an amount sufficient to enhance the solubility of the ketorolac free acid or salt thereof in the water. The ketorolac may be present as the free acid or salt. The formulation may be partially pressurized. Preferably, the ketorolac or pharmaceutically acceptable salt or free acid thereof is present in the formulation at a concentration of 0.001%-80%. Preferably, the ketorolac or pharmaceutically acceptable salt or free acid thereof is present at a concentration of 0.01%-20% by weight.

Preferably, the polar organic solvent is an alcohol. The alcohol may include, but is not limited to, ethanol, propylene glycol, glycerol, polyethylene glycol and mixtures thereof. Preferably, the alcohol is ethanol. Preferably, the polar organic solvent is present in an amount of 0-90% w/w.

The formulation may be buffered. The buffer may include citrate or phosphate buffer. Preferably, the formulation has a pH of less than 9. More preferably, the formulation has a pH of about 7.0. The formulation may further comprise a sweetener. Preferably, the sweetener is mannitol, saccharin, and/or saccharin sodium. The formulation may also further comprise a flavoring agent. Preferably, the flavoring agent is mint.

In yet another embodiment, the invention provides a method of providing fast relief from the symptoms of pain, comprising administering to a subject in need thereof a pharmaceutically effective amount of ketorolac, by spraying the ketorolac onto the subject's sublingual mucosa. The ketorolac may be in the form of a ketorolac free acid or tromethamine salt, or any acceptable salt dissolved in an ethanolic solution.

In another embodiment, the invention provides a method of providing fast relief from the symptoms of pain comprising administering to a subject in need thereof a liquid spray formulation, comprising (i) ketorolac or pharmaceutically acceptable salt or free acid thereof, (ii) buffered water; and (iii) a polar organic solvent is provided. The polar organic solvent is present in an amount sufficient to enhance the solubility of the ketorolac free acid or salt thereof in the water. The formulation is sprayed onto the subject's sublingual mucosa.

In another embodiment, the invention provides a metered dose dispensing system for the administration of a liquid spray formulation, which comprises (i) ketorolac or pharmaceutically acceptable salt or free acid thereof, (ii) buffered water; and (iii) a polar organic solvent is provided.

The polar organic solvent is present in an amount sufficient to enhance the solubility of the ketorolac free acid or salt thereof in the water. The metered dose dispensing system comprises a sealed container fitted with a metering pump, an actuator and a channeling device. Preferably, the metered dose dispensing system contains a metering chamber which is adapted for dispensation with the container in the upright orientation, and wherein the metering chamber is in communication with the formulation by means of a dip-tube.

In another embodiment the, the invention provides a method of providing relief from pain, comprising administering to a subject in need thereof a liquid spray formulation, which comprises (i) ketorolac or pharmaceutically acceptable salt or free acid thereof, (ii) buffered water; and (iii) a polar organic solvent is provided. The polar organic solvent is present in an amount sufficient to enhance the solubility of the ketorolac free acid or salt thereof in the water. The formulation is sprayed onto the subject's sublingual mucosa. Preferably, the relief from pain is achieved within 20 minutes. More preferably, the relief from pain is achieved within 5 minutes.

These and other embodiments of the invention are described herein below or are evident to persons of ordinary skill in the art based on the following disclosures.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

This invention, as defined in the claims, can be better understood with reference to the following drawings:

FIG. 1 illustrates the mean plasma ketorolac levels following sublingual spray (3 mg/kg) and intravenous administration at 0.5 mg/kg single dose in rabbits (n=3).

In the following description of the illustrated embodiments, references are made to the accompanying drawings, which form a part hereof, and in which is shown by way of illustration various embodiments in which the invention may be practiced. It is to be understood that other embodiments may be utilized and structural and functional changes may be made without departing from the scope of the present invention.

### DETAILED DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All references, publications, patents, patent applications, and commercial materials mentioned herein are incorporated herein by reference for all purposes including for describing and disclosing the cell lines, vectors, and methodologies which are reported in the publications which might be used in connection with the invention. Nothing herein is to be construed as an admission that the invention is not entitled to antedate such disclosure by virtue of prior invention.

In order to provide a clear and consistent understanding of the specification and claims, including the scope to be given such terms, the following definitions are provided:

The term “administration” of the pharmaceutically active compounds and the pharmaceutical compositions defined herein includes sublingual spray application.

“Ameliorate” or “amelioration” means a lessening of the detrimental effect or severity of the disease in the subject receiving therapy, the severity of the response being determined by means that are well known in the art.

By “compatible” herein is meant that the components of the compositions which comprise the present invention are capable of being commingled without interacting in a manner which would substantially decrease the efficacy of the pharmaceutically active compound under ordinary use conditions.

The terms “effective amount” or “pharmaceutically effective amount” refer to a nontoxic but sufficient amount of the agent to provide the desired biological result. That result can be reduction and/or alleviation of the signs, symptoms, or causes of a disease, such as neural diseases and malignant hyperthermia, or any other desired alteration of a biological system. Such amounts are described below. An appropriate “effective” amount in any individual case may be determined by one of ordinary skill in the art using routine experimentation.

As used herein, the term “excipient” means the substances used to formulate active pharmaceutical ingredients (API) into pharmaceutical formulations; in a preferred embodiment, an excipient does not lower or interfere with the primary therapeutic effect of the API. Preferably, an excipient is therapeutically inert. The term “excipient” encompasses carriers, diluents, vehicles, solubilizers, stabilizers, bulking agents, acidic or basic pH-adjusting agents and preservatives. Excipients can also be those substances present in a pharmaceutical formulation as an indirect or unintended result of the manufacturing process. Preferably, excipients are approved for or considered to be safe for human and animal administration, i.e., GRAS substances (generally regarded as safe). GRAS substances are listed by the Food and Drug administration in the Code of Federal Regulations (CFR) at 21 CFR 182 and 21 CFR 184, incorporated herein by reference.

As used herein, the terms “formulate” refers to the preparation of a drug in a form suitable for administration to a mammalian patient, preferably a human. Thus, “formulation” can include the addition of pharmaceutically acceptable excipients, diluents, or carriers and pH adjusting agents.

The term “ketorolac” as used herein includes the free acid form of this compound as well as pharmacologically acceptable salts thereof. In one embodiment, the pharmaceutical composition comprises ketorolac tromethamine; however, other pharmacologically acceptable salts thereof can be utilized as well. For the purposes of the present invention, ketorolac can be a racemic mixture, or pure ketorolac enantiomers (i.e. either (−)-ketorolac or (+)-ketorolac), or a non-racemic mixtures of ketorolac enantiomers (i.e. mixtures where the molar ratio of one enantiomer is greater than, or less than, that of the other).

The term “permeation enhancer” or “penetration enhancer” as used herein refers to an agent that improves the rate of transport of a pharmacologically active agent (e.g., ketorolac) across the sublingual mucosal surface. Typically a penetration enhancer increases the permeability of mucosal tissue to a pharmacologically active agent. Penetration enhancers, for example, increase the rate at which the pharmacologically active agent permeates through membranes and enters the bloodstream. Enhanced permeation effected through the use of penetration enhancers can be observed, for example, by measuring the flux of the pharmacologically active agent across animal or human membranes as described in the Examples herein below. An “effective” amount of a permeation enhancer as used herein means an amount that will provide a desired increase in mucosal membranes permeability to provide, for example, the desired depth of penetration of a selected compound, rate of administration of the compound, and amount of compound delivered.

“Optional” or “optionally” means that the subsequently described event or circumstance may, but need not, occur, and that the description includes instances where the event or circumstance occurs and instances in which it does not.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

As used herein, a “pharmaceutically acceptable carrier” is a material that is nontoxic and generally inert and does not affect the functionality of the active ingredients adversely. Examples of pharmaceutically acceptable carriers are well known and they are sometimes referred to as diluents, vehicles or excipients. The carriers may be organic or inorganic in nature. In addition, the formulation may contain additives such as flavoring agents, coloring agents, thickening or gelling agents, emulsifiers, wetting agents, buffers, stabilizers, and preservatives such as antioxidants.

“Pharmaceutically acceptable salt” refers to pharmaceutically acceptable salts of ketorolac which salts are derived from a variety of organic and inorganic counter ions well known in the art and include, by way of example only, tromethamine, sodium, potassium, calcium, magnesium, ammonium, tetraalkylammonium, and the like.

The term “pharmaceutical composition” as used herein shall mean a composition that is made under conditions such that it is suitable for administration to humans, e.g., it is made under GMP conditions and contains pharmaceutically acceptable excipients, e.g., without limitation, stabilizers, pH adjusting agents, bulking agents, buffers, carriers, diluents, vehicles, solubilizers, and preservatives.

The liquid carrier or vehicle can be a solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. The prevention of the growth of microorganisms can be accomplished by various antibacterial and antifungal agents, for example, parabens, chlorobutanol, phenol, sorbic acid, thimerosal, and the like.

As used herein, the term “subject” encompasses mammals and non-mammals. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or sex.

The term “sublingual administration” refers to the mode of administration of a medicament to the tissue under the tongue.

The term “suitable for sublingual administration” refers to any mode of administration of a medicament to the tissue under the tongue. For example, a spray may be used.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Treating” or “treatment” of a disease includes: (1) preventing the disease, i.e., causing the clinical symptoms of the disease not to develop in a mammal that may be exposed to or predisposed to the disease but does not yet experience or display symptoms of the disease, (2) inhibiting the disease, i.e., arresting or reducing the development of the disease or its clinical symptoms, or (3) relieving the disease, i.e., causing regression of the disease or its clinical symptoms.

General

The present invention provides for compositions and methods for the delivery of ketorolac, or pharmaceutically acceptable salt thereof, to a patient in need of such treatment. As described above, this invention provides a ketorolac spray for sublingual administration, used in the treatment and amelioration of pain and symptoms caused by other conditions. Also provided are methods of treatment, amelioration and inhibition of these conditions and symptoms, as well as a metered dosage system for administration of the spray.

The present invention is directed to the administration of ketorolac by the sublingual route. Sublingual administration has the potential for providing an alternative to intravenous and oral dosing for rapid delivery of drugs to the systemic circulation. Thus, sublingual drug delivery by-passes gastrointestinal and hepatic pre-systemic elimination, and is a useful form of drug delivery for patients with swallowing problems. This is especially effective if a patient is experiencing nausea and emesis, and cannot readily swallow an oral dosage form.

Ketorolac Spray

The present invention provides a pharmaceutical composition comprising ketorolac, provided for sublingual administration. The present invention also provides a liquid formulation for administering ketorolac to the sublingual mucosa (i.e., under the tongue) by a spray. This formulation preferably comprises ketorolac or acceptable salt or free acid thereof, buffered water, and a polar organic solvent.

In general, the compounds of the subject invention will be administered in a therapeutically effective amount by sublingual administration. Preferably, the formulation is administered as a liquid spray composition. Such compositions are prepared in a manner well known in the pharmaceutical art. Preferably, the spray is administered directly to the sublingual mucosa.

In one embodiment, the ketorolac is a racemic mixture, pure ketorolac enantiomers (i.e. either (−)-ketorolac or (+)-ketorolac), or a non-racemic mixture of ketorolac enantiomers (i.e. mixtures where the molar ratio of one enantiomer is greater than, or less than, that of the other). In another embodiment, the ketorolac is in the form of ketorolac free acid or tromethamine salt.

The composition for the sublingual spray formulation comprising ketorolac or pharmaceutically acceptable salt thereof provided in racemic mixture, pure ketorolac enantiomers (i.e. either (−)-ketorolac or (+)-ketorolac), or a non-racemic mixtures of ketorolac enantiomers (i.e. mixtures where the molar ratio of one enantiomer is greater than, or less than, that of the other).

In another embodiment, the present invention further provides a liquid spray formulation, comprising ketorolac or pharmaceutically acceptable salt or free acid thereof and a pharmaceutically acceptable carrier.

In one embodiment, the carrier is water, buffered water, organic solvent, or combinations thereof. In another embodiment, the organic solvent is present in an amount sufficient to enhance the solubility of the ketorolac free acid or salt thereof in the water.

In some embodiments, the ketorolac or pharmaceutically acceptable salt or free acid thereof is present at a concentration of 0.001% -80%. Preferably, the ketorolac or pharmaceutically acceptable salt or free acid thereof is present at a concentration of 0.01% -20% by weight.

In another embodiment, the organic solvent is an alcohol. The alcohol may include, but is not limited to, ethanol, propylene glycol, glycerol, polyethylene glycol and mixtures thereof. In another embodiment, the alcohol is ethanol. In another embodiment, the organic solvent is present in an amount of 0-90% by weight.

In another embodiment, the carrier of the dosage unit is preferably an aqueous solution. Further, the aqueous solution can be selected from the group including aqueous gels, aqueous suspensions, aqueous liposomal dispersions, aqueous emulsions, aqueous microemulsions, aqueous nanoparticles and combinations thereof.

In another embodiment, the carrier of the dosage unit is a nonaqueous organic solution. The non-aqueous organic solution can be selected from a group including non-aqueous gels, non-aqueous suspensions, non-aqueous liposomal dispersions, nonaqueous emulsions, non-aqueous microemulsions, non-aqueous nanoparticles and combinations thereof.

In another embodiment, the carrier of the dosage unit is a combination of an aqueous solution and a non-aqueous solution. The formulation may be partially pressurized.

In another embodiment, the pharmaceutical composition can include a buffer to maintain the pH of the formulation. The pharmaceutical composition can further include one or more pharmaceutical excipients and even further include a pharmaceutically acceptable preservative.

In another embodiment, the buffer of the sublingual spray formulation may be, but not limited to, acetate, citrate, prolamine, carbonate and phosphate buffers and combinations thereof.

In another embodiment, the composition comprises a permeation enhancer. In another embodiment, the permeation enhancer is selected from the group consisting of a bile salt, sodium dodecyl sulfate, dimethyl sulfoxide, sodium lauryl sulfate, a derivative of a saturated or unsaturated fatty acid, a surfactant, synthetic permeation enhancer, a bile salt analog, a derivative of a bile salt and combinations thereof.

In another embodiment, the composition comprises a thickening agent which can be, but not limited to, methyl cellulose, xanthan gum, carboxymethyl cellulose, hydroxypropyl cellulose, carbomer, polyvinyl alcohol, alginates, acacia, chitosans and combinations thereof.

In another embodiment, the formulation may further comprise a sweetener suitable for sublingual delivery systems. In one embodiment, the sweetener may be, but is not limited to, mannitol, saccharin or saccharin sodium.

In another embodiment, the formulation may further comprise a flavoring agent. In one embodiment, the flavoring agent is mint.

In another embodiment, the formulation may further comprise a bioadhesive. The bioadhesive can be, but is not limited to, polyacrylics, cellulosics, hyaluronates, chitosan, polyvinyl pyrrolidone, and gelatin.

In another embodiment, the invention further provides a liquid spray formulation, comprising ketorolac or pharmaceutically acceptable salt or free acid thereof, in an amount of 0.001%-80%. Preferably, the ketorolac or pharmaceutically acceptable salt or free acid thereof is present at a concentration of 0.01%-20% by weight.

In another embodiment, the sublingual spray formulation can be delivered with conventional spray device or a metered dose dispensing system for the administration of a liquid spray formulation, comprising ketorolac or a pharmaceutically acceptable salt and a pharmaceutically acceptable carrier.

In another embodiment, the present invention further provides a method of providing fast relief from pain, comprising administering to a subject in need thereof a pharmaceutically effective amount of ketorolac, by spraying the ketorolac onto the subject's sublingual mucosa under the tongue.

As described herein, this invention provides a novel method for delivering active agents comprise ketorolac free acid, ketorolac tromethamine, or any pharmaceutically acceptable salts to the systemic circulation through sublingual spray formulations. More specifically, the invention relates to a ketorolac liquid spray formulation for administration onto the sublingual mucosa under the tongue, to provide a rapid response in the treatment of pain, especially postoperative pain and migraine headache to a subject in need of this treatment.

The compositions and methods of the present invention provide a rapid, reliable, safe, effective and convenient method for delivering ketorolac to the sublingual mucosa under the tongue to treat postoperative pain and migraine headache comprising a spray formulation.

In another embodiment, the present invention concerns the administration of ketorolac to the sublingual mucosa under the tongue via spray formulations. “Ketorolac” refers to the compound: 5-benzoyl-2,3-dihydro-1H-Pyrrolizine-1-carboxylic acid, and has the following formula:

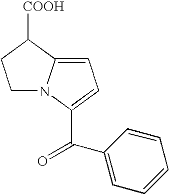

In one embodiment, the ketorolac therapeutic effect is achieved to a degree sufficient to cause a relief of pain, especially the one associated with postoperative pain and migraine headache by the sublingual administration of ketorolac through liquid spray formulation so as to maintain an adequate plasma concentration of ketorolac. In another embodiment, the amount of ketorolac administered is an amount sufficient to cause therapeutic effect but is low enough not to cause substantial intolerable adverse side effects. As used herein, “substantial intolerable adverse side effects” include those effects caused by either the delivery system or the drug which are incompatible with the health of the user or which are so unpleasant as to discourage the continued use of the composition.

In another embodiment, the ketorolac is present in the compositions and formulations in an amount sufficient to treat postoperative pain and migraine headache. The active compound is effective over a wide dosage range and is generally administered in a pharmaceutically or therapeutically effective amount. In another embodiment, the therapeutic dosage of the compounds of the present invention will vary according to, for example, the particular use for which the treatment is made, the manner of administration of the compound, the health and condition of the patient, and the judgment of the prescribing physician. In another embodiment, the ketorolac or pharmaceutically acceptable salt or free acid thereof is present at a concentration of 0.001%-80%.

Despite the overall popularity of other delivery methods, oral transmucosal delivery through sublingual mucosa under the tongue is particularly advantageous delivery system. One of the advantages of sublingual administration is that it is non-invasive. Furthermore, transmucosal delivery generally has better patient compliance, less risk of infection and lower cost than invasive procedures such as injection and implantation. Furthermore, the liquid spray formulation is advantageous in producing shorter onset time, i.e., the time from administration to therapeutic effect, than the oral delivery. A drug absorbed via the sublingual mucosa will also avoid first pass metabolism, in which the drug is metabolized in the gastrointestinal tract and liver. Similarly, a drug absorbed via the sublingual mucosa will avoid the variability in gastric emptying time commonly observed in patients with proximal gastrointestinal motility syndromes, allowing for greater predictability in obtaining therapeutic blood levels. Such transmucosal delivery systems are simple and dosage forms can be administered by the caregiver or the patient with minimal discomfort.

The composition may further include additional pharmaceutical ingredients to provide desirable characteristics, such as pleasing qualities, improved taste, and the like, to otherwise render the dosage formulation more likely to be administered by the patient. Examples of desirable ingredients include, without limitation, penetration enhancers, adhesives, antioxidants, buffers, colorants, flavorings agents, solvents and co-solvents, suspending agents, sweetening agents, and diluents. The ingredients may also include preservatives, emulsifying agents, plasticizers, surfactants, tonicity agents, viscosity increasing agents and combinations thereof. Examples of useful additives include, without limitation, propylene glycol, polyethylene glycol, orange, cherry, mint, and strawberry flavors and other commonly utilized ingredients.

In another embodiment, the composition according to the present invention includes a viscosity increasing agents such as, but not limited to, natural gums, methylcellulose and derivatives, acrylic polymers (carbopol) and vinyl polymers (polyvinylpyrrolidone).

In another embodiment, the components of the composition are formulated in any suitable liquid spray dosage form to deliver ketorolac to the sublingual tissue under the tongue.

In another embodiment, the sublingual spray formulations of the present invention can take various forms including, but not limited to, aqueous solutions, non-aqueous solutions and combinations thereof. Aqueous solutions include, for example, aqueous gels, aqueous suspensions, aqueous liposomal dispersions, aqueous emulsions, aqueous microemulsions and combinations thereof. Non-aqueous solutions include, for example, non-aqueous gels, non-aqueous suspensions, non-aqueous liposomal dispersions, non-aqueous emulsions, non-aqueous microemulsions and combinations thereof.

In another embodiment, the pH of the compositions is maintained from about 3.0 to about 10.0. Compositions having a pH of less than about 3.0 or greater than about 10.0 can increase the risk of irritating the mucosal membranes in the sublingual region of a recipient. In another embodiment, the pH of the compositions is maintained from about 3.0 to about 7.0.

In another embodiment, preservatives are added to the present compositions. Suitable preservatives that can be used with the present compositions include benzyl alcohol, parabens, thimerosal, chlorobutanol and benzalkonium chloride and preferably benzalkonium chloride is used. In another embodiment, the preservative are present in a composition in a concentration of up to about 2% by weight. The exact concentration of the preservative, however, will vary depending upon the intended use and can be easily ascertained by one skilled in the art.

In another embodiment, the compositions of the invention can be formulated so as to provide quick, sustained or delayed release of the active ingredient after administration to the patient by employing procedures known in the art. In another embodiment, the formulation may also comprise a muco-adherent to increase the residence time on the mucosa; including chitosan, polyvinyl pyrrolidone, or gelatin.

In another embodiment, the formulation may further comprise a moisturizing agent, such as propylene glycol, or polyethylene glycol. The formulation may further comprise an antioxidant, such as butylated hydroxyltoluene, ascorbic acid, alkyl gallates, or tocopherols. The formulation may further comprise an ionic or nonionic surfactant, such as sodium lauryl sulfate, or sorbitan esters.

In another embodiment, the formulation may further comprise a co-solvent. In another embodiment, the organic solvent is an alcohol. In another embodiment, the alcohol may comprise, but is not limited to, ethanol, propylene glycol, glycerol, polyethylene glycol and mixtures thereof. More preferably, the alcohol is ethanol. In another embodiment, the organic solvent is present in an amount of 0-90% w/w.

In another embodiment, the composition in this invention can be administered as a spray for sublingual administration; the ketorolac is preferably in the form of the ketorolac free acid or tromethamine salt, or any pharmaceutically acceptable salt.

In another embodiment, the present invention provides for the compositions as described above which are administered to the sublingual tissue under the tongue to a mammal to treat postoperative pain and migraine headache. In another embodiment, the formulation is administered as a spray. In another embodiment, the spray is administered directly to the sublingual mucosa, i.e., the formulation is sprayed directly onto the tissue under the patient's tongue. By administering the ketorolac directly to the sublingual mucosa, the patient can experience fast and even immediate relief, while still maintaining a high level of bioavailability. A patient suffering from these symptoms can feel relief within 1-10 minutes, with a maximum concentration of the drug being reached within 20 minutes or faster. Thus, someone suffering from migraine pain can experience an immediate relief.

In one embodiment of the present invention, the composition administration will be through a spray system. Such system can include a conventional spray device or a metered dose dispensing systems known in the art, providing a convenient way to confirm that each spray dose is identical in amount.

In another embodiment, the ketorolac is present in the compositions and formulations in an amount sufficient to prevent, treat, relieve, and/or inhibit pain and other conditions with similar symptoms of pain. The active compound is effective over a wide dosage range and is generally administered in a pharmaceutically or therapeutically effective amount. The therapeutic dosage of the compounds of the present invention will vary according to, for example, the particular use for which the treatment is made, the manner of administration of the compound, the health and condition of the patient, and the judgment of the prescribing physician. In another embodiment, the actual amount of the compound, i.e., ketorolac and acceptable salts and free bases thereof, will depend on a number of factors, such as the severity of the disease, i.e., the condition or disease and symptoms, the age and relative health of the subject, the potency of the compound used the route and form of administration, and other factors.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population).

The dose ratio between toxic and therapeutic effects is the therapeutic index and it can be expressed as the ratio LD₅₀/ED₅₀. Compounds that exhibit large therapeutic indices are preferred.

The data obtained from cell culture assays and animal studies can be used in future formulating of a range of dosage for use in humans and other animal patients. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range which includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The amount administered to the patient will vary depending upon what is being administered, the purpose of the administration, such as prophylaxis versus therapy, the state of the patient, the manner of administration, and the like. In therapeutic applications, compositions are administered to a patient already suffering from symptoms and/or a condition in an amount sufficient to cure or at least partially arrest the symptoms and complications. An amount adequate to accomplish this is defined as “therapeutically effective dose.” Amounts effective for this use will depend on the age, weight and general condition of the subject/patient, and the like.

In another embodiment, the polar organic solvent is present in an amount which will enhance the solubility of the ketorolac in water. In another embodiment, the organic solvents include, but are not limited to, alcohols, such as ethanol, propylene glycol, glycerol, polyethylene glycol and mixtures thereof. In another embodiment, the polar organic solvent may be present in the formulation in an amount of about 0-90% w/w. In another embodiment, the formulation may be buffered, as appropriate.

In another embodiment, the compositions administered to a subject are in the form of pharmaceutical compositions. These compositions may be sterilized by conventional sterilization techniques, or may be sterile filtered. It will be understood that use of certain of the foregoing excipients, carriers, or stabilizers will result in the formation of pharmaceutical salts. When employed as pharmaceuticals, the compounds of the subject invention are usually administered in the form of pharmaceutical compositions. In another embodiment, the invention also includes pharmaceutical compositions, which contain as the active ingredient, one or more of the compounds of the subject invention above, associated with one or more pharmaceutically acceptable carriers or excipients. In another embodiment, the excipient employed is typically one suitable for administration to human subjects or other mammals. In making the compositions of this invention, the active ingredient is usually mixed with an excipient, diluted by an excipient. When the excipient serves as a diluent, it can be a solid, semi-solid, or liquid material, which acts as a vehicle, carrier or medium for the active ingredient.

In another embodiment, the compositions of the invention can be formulated so as to provide quick, sustained or delayed release of the active ingredient after administration to the patient by employing procedures known in the art. In another embodiment, the ketorolac formulation may further comprise a sweetener, such a mannitol, saccharin, and saccharin sodium. In another embodiment, the formulation may further comprise a flavoring agent, such as menthol.

To assist in the speed of efficacy and bioavailability, the formulation may also comprise a penetration enhancer. Preferable, the permeation enhancer is selected from the group consisting of a bile salt, sodium dodecyl sulfate, dimethyl sulfoxide, sodium lauryl sulfate, a derivative of a saturated or unsaturated fatty acid, a surfactant, synthetic permeation enhancer, a bile salt analog, a derivative of a bile salt and combinations thereof.

In another embodiment, the formulation may further comprise a moisturizing agent, such as propylene glycol, or polyethylene glycol. In another embodiment, the formulation may further comprise a preservative such as sodium metabisulphite, benzalkonium chloride, or ethanol. In another embodiment, the formulation may further comprise an antioxidant, such as butylated hydroxyltoluene, ascorbic acid, alkyl gallates, or tocopherols. In another embodiment, the formulation may further comprise an ionic or nonionic surfactant, such as sodium lauryl sulfate, or sorbitan esters.

In another embodiment, the liquid forms in which the compositions of the present invention may be incorporated for administration by spray include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles. A spray formulation may be prepared by methods well known in the art.

According to one embodiment of the invention, the compound may be administered alone, or in combination with any other medicament. Such medicaments include, but are not limited to, analgesic agents. When administered in combination, the compounds may be administered in the same formulation as these other compounds or compositions, or in a separate formulation. When administered in combination, the ketorolac spray may be administered prior to, following, or concurrently with the other compounds and compositions.

When administered as a spray for sublingual administration, the ketorolac is preferably in the form of ketorolac free acid or tromethamine salt, or any acceptable salt dissolved in an ethanolic solution. Suitable methods and formulations for use in the present invention are found in REMINGTON'S PHARMACEUTICAL SCIENCES, Mace Publishing Company, Philadelphia, Pa., 17th ed. (1985).

Methods of Treatment and Amelioration

In another embodiment, the present invention provides methods of treating, inhibiting and/or preventing pain.

In another embodiment, the formulation is preferably administered as a spray. Preferably, the spray is administered directly to the sublingual mucosa, i.e., the formulation is sprayed directly onto the tissue under the patient's tongue. By administering the ketorolac directly to the sublingual mucosa, the patient can experience fast and even immediate relief, while still maintaining a high level of bioavailability. A patient suffering from these symptoms can feel relief within 1-5 minutes, with a maximum concentration of the drug being reached within 20 minutes or faster.

Metered Dosage System

The present invention further provides a device and system for administering the ketorolac spray. Such a system can include a metered dose dispensing system, providing a convenient way to confirm that each spray dose is identical in amount. The metered dosage system may comprise a sealed container, which is fitted with a metering pump, an actuator and a channeling device. The metered dosage system may further contain a metering chamber adapted for dispensation with the container in the upright orientation. The metering chamber would be placed in communication with the formulation by means of a dip-tube.

Metered dosage systems well known in the art may be used. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In another embodiment, the composition comprises a gelling or thickening agent(s). Exemplary gelling agents include, but are not limited to, carbomer, carboxyethylene or polyacrylic acid such as carbomer 980 or 940 NF, 981 or 941 NF, 1382 or 1342 NF, 5984 or 934 NF, ETD 2020, 2050, 934P NF, 971P NF, 974P NF, polycarbophils such as NOVEON AA-1, NOVEON CA1/CA2, carbomer copopolymers such as PEMULEN TR1 NF or PEMULEN TR2 NF, carbomer interpolymers such as CARBOPOL ETD 2020 NF, CARBOPOL ETD 2050 NF, CARBOPOL ULTRA EZ 10, etc.; cellulose derivatives such as ethylcellulose, hydroxypropylmethylcellulose (HPMC), ethyl-hydroxyethylcellulose (EHEC), carboxymethylcellulose (CMC), hydroxypropylcellulose (HPC), hydroxyethylcellulose (HEC), etc.; natural gums such as arabic, xanthan, guar gums, alginates, etc.; polyvinylpyrrolidone derivatives; polyoxyethylene polyoxypropylene copolymers, etc; others like chitosan, polyvinyl alcohols, pectins, veegum grades, and the like. Other suitable gelling agents to apply the present invention include, but are not limited to, carbomers. Alternatively, other gelling agents or viscosant known by those skilled in the art may also be used. The gelling agent or thickener is present from about 0.2 to about 30% w/w depending on the type of polymer, as known by one skilled in the art. A preferred concentration range of the gelling agent(s), for example, hydroxypropyl cellulose or carbomer, is a concentration of between about 0.5 and about 5 weight percent, more preferred is a concentration of between about 1 and about 3 weight percent.

In another embodiment, the composition comprises a permeation enhancer (penetration enhancer). Permeation enhancers include, but are not limited to, sulfoxides such as dimethylsulfoxide and decylmethylsulfoxide; surfactants such as sodium laurate, sodium lauryl sulfate, cetyltrimethylammonium bromide, benzalkonium chloride, poloxamer (231, 182, 184), tween (20, 40, 60, 80) and lecithin; the 1-substituted azacycloheptan-2-ones, particularly 1-n-dodecylcyclazacycloheptan-2-one; fatty alcohols such as lauryl alcohol, myristyl alcohol, oleyl alcohol and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate, amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine, terpenes; alkanones, and organic acids, particularly salicylic acid and salicylates, citric acid and succinic acid. As noted earlier herein, “Percutaneous Penetration Enhancers”, eds. Smith et al. (CRC Press, 1995), which is incorporated herein by reference thereto, provides an excellent overview of the field and further information concerning possible secondary enhancers for use in conjunction with the present invention. More permeation enhancer(s) suitable to be used with the present invention may be known by those skilled in the art. In another embodiment, the permeation enhancer is present from about 0.1 to about 30% w/w depending on the type of compound. In another embodiment, the permeation enhancers are fatty alcohols and fatty acids. In another embodiment, the permeation enhancers are fatty alcohols. In another embodiment, the fatty alcohols have the formula the CH₃(CH₂)ₙ(CH)ₘCH₂OH wherein n ranges from (8-m) to (16-m) and m=0-2. In another embodiment, the concentration range of the penetration enhancer(s) is, depending on the type of permeation enhancer, a concentration of between about 0.1 and about 10 weight percent, as known by one skilled in the art. In one preferred embodiment, the penetration enhancer comprises myristyl alcohol in a concentration of between about 0.1 and about 2 weight percent.

In some embodiments, the permeation enhancer is chosen from: a bile salt, sodium dodecyl sulfate, dimethyl sulfoxide, sodium lauryl sulfate, a derivative of a saturated or unsaturated fatty acid, a surfactant, a bile salt analog, and a derivative of a bile salt. In some embodiments the permeation enhancer is a synthetic permeation enhancer.

In another embodiment, the composition comprises antioxidant(s), for example, tocopherol and derivatives, ascorbic acid and derivatives, butylated hydroxyanisole, butylated hydroxytoluene, fumaric acid, malic acid, propyl gallate, sodium metabisulfite and derivatives, is a concentration of about 0.01 to about 5 weight percent; in another embodiment, is a concentration of about 0.1 to about 0.5 weight percent, depending on the type of antioxidant used, as known by the one skilled in the art.

In another embodiment, the composition comprises buffering agent(s), for example, carbonate buffers, citrate buffers, phosphate buffers, acetate buffers, hydrochloric acid, lactic acid, tartaric acid, inorganic and organic bases, is a concentration of about 1 to about 10 weight percent, more preferred is a concentration of about 2 to about 5 weight percent, depending on the type of buffering agent(s) used, as known by the one skilled in the art. The preferred concentration range of said buffering agents are those enabling design of compositions having a pH close to the physiologic pH of the mucosal membranes, between about pH 2.0 and about pH 10.0, preferably between about pH 3.0 and pH 7.0. Concentrations of the buffering agent(s) may vary, however, as known by the one skilled in the art. The buffering agent may replace up to 100% of the water amount within the composition.

In another embodiment, the pharmaceutical formulation of the present invention may also further include preservatives such as benzalkonium chloride and derivatives, benzoic acid, benzyl alcohol and derivatives, bronopol, parabens, centrimide, chlorhexidine, cresol and derivatives, imidurea, phenol, phenoxyethanol, phenylethyl alcohol, phenylmercuric salts, thimerosal, sorbic acid and derivatives. The preservative is present from about 0.01 to about 10% w/w depending on the type of compound used, as known by the one skilled in the art.

In another embodiment, the pharmaceutical formulation of the present invention may also further include humectants, sequestering agents, moisturizers, surfactants, emollients, colorants, fragrances, flavors, or any combination thereof.

In some embodiments, the dosage form is a liquid formulation, comprising: ketorolac or pharmaceutically acceptable salt thereof, aqueous solvent; and a polar organic solvent, wherein the polar organic solvent is present in an amount sufficient to enhance the solubility of the ketorolac free acid or salt thereof in the water.

In one embodiment, a gel formulation of the present invention comprises a therapeutically effective amount of a ketorolac, or a pharmaceutically acceptable salt or derivative thereof, of between about 0.01 to about 5 weight percent. In another embodiment, the primary vehicle may comprise between about 10 to about 60 weight percent of water, between about 30 to about 70 weight percent ethanol, between about 15 and about 60 weight percent of a 10:1 to 1:10 (weight to weight) mixture of diethylene glycol mono ethyl ether and propylene glycol, and between about 0.1 and about 2 weight percent of lauryl alcohol, myristyl alcohol, oleyl alcohol, lauric acid, myristic acid, or oleic acid. In another embodiment, the primary vehicle may be gellified with between about 0.5 and about 5 weight percent of hydroxypropylcellulose. In another embodiment, the apparent pH of the gel is between about pH 2.0 and about pH 10.0, or preferably between about pH 3.0 and pH 7.0.

In another embodiment, the delivery system of the pharmaceutical composition can include a buffer to maintain the pH of the formulation and a pharmaceutically acceptable thickening agent. The pharmaceutical composition can further include one or more pharmaceutical excipients and even further include a pharmaceutically acceptable preservative.

In another embodiment, the buffer of the delivery system can be selected from the group including acetate, citrate, prolamine, carbonate and phosphate buffers.

In another embodiment, the thickening agent of the delivery system can be selected from the group including methyl cellulose, xanthan gum, carboxymethyl cellulose, hydroxypropyl cellulose, carbomer, polyvinyl alcohol, alginates, acacia, chitosans and combinations thereof.

In another embodiment, the formulation may further comprise a sweetener suitable for sublingual spray delivery systems. In another embodiment, the sweetener may be, but is not limited to, mannitol, saccharin or saccharin sodium. In another embodiment, the formulation may further comprise a flavoring agent. In another embodiment, the flavoring agent is mint. In another embodiment, the formulation may further comprise a thickening agent. In another embodiment, the thickening agent may be, but is not limited to, methyl cellulose, xanthan gum, carboxymethyl cellulose, hydroxypropyl cellulose, carbomer, polyvinyl alcohol, alginates, acacia, chitosans and combinations thereof.

In some embodiments, the carrier of the transmucosal dosage unit is preferably an aqueous solution. In another embodiment, the aqueous solution can be selected from the group including aqueous gels, aqueous suspensions, aqueous liposomal dispersions, aqueous emulsions, aqueous microemulsions, aqueous nanoparticles and combinations thereof.

In another embodiment, the carrier of the dosage unit is a nonaqueous solution. In another embodiment, the non-aqueous solution can be selected from a group including non-aqueous gels, non-aqueous suspensions, non-aqueous liposomal dispersions, nonaqueous emulsions, non-aqueous microemulsions, non-aqueous nanoparticles and combinations thereof.

In another embodiment, the carrier of the dosage unit can also be a combination of an aqueous solution and a non-aqueous solution. The formulation may be partially pressurized. In yet another aspect the present invention comprises a composition for pharmaceutical drug delivery, comprising a therapeutically effective amount of ketorolac, or a pharmaceutically acceptable salt, in a hydroalcoholic vehicle comprising water, a short chain alcohol, a monoalkyl ether of diethylene glycol, a pharmaceutically acceptable glycol, and an optional fatty permeation enhancer. These compositions for pharmaceutical delivery may include further components as described herein, for example, the hydroalcoholic vehicle may further comprise a co-solvent(s), a penetration enhancer(s), a buffering agent(s), a preservative(s), an emollient(s), an humectant(s), and/or a gelling agent(s). Such compositions may be formulated in a variety of ways including wherein the hydroalcoholic vehicle is slightly gellified. These compositions may be used, for example, for transmucosal applications including spray application to sublingual tissues.

In a further aspect, the present invention includes methods of manufacturing the compositions described herein for pharmaceutical drug delivery. In one embodiment, the method of manufacturing comprises mixing the components to yield a homogeneous gel, wherein the pH of the gel is between about pH 3 and about pH 7 (exemplary components include, but are not limited to the following: a therapeutically effective amount of ketorolac, or a pharmaceutically acceptable salt thereof, a primary vehicle comprising water, at least one short-chain alcohol, a monoalkyl ether of diethylene glycol, a pharmaceutically acceptable glycol, an optional fatty permeation enhancer). These methods may include addition of further components as described herein, for example, the hydroalcoholic vehicle may further comprise co-solvent(s), penetration enhancer(s), buffering agent(s), preservative(s), emollient(s), humectant(s), and/or gelling agent(s). The method provides a gel suitable for pharmaceutical sublingual delivery of ketorolac.

The present invention further includes methods for administering a composition of the present invention to a subject in need thereof. Compositions of the present invention comprising ketorolac can be employed, for example, for the treatment of a variety of conditions and/or disease states which have been historically treated by oral doses of ketorolac.

The ketorolac compositions of the present invention may be self-applied by a subject in need of treatment or the composition may be applied by a care-giver or health care professional.

The compositions of the invention can be formulated so as to provide fast and/or sustained release of the active ingredient after administration to the patient by employing procedures known in the art.

The following examples are offered to illustrate this invention and are not to be construed in any way as limiting the scope of this invention.

### EXAMPLES

The following Examples are provided to illustrate certain aspects of the present invention and to aid those of skill in the art in practicing the invention. These Examples are in no way to be considered to limit the scope of the invention in any manner.

### Example 1

Ketorolac acid 10.0%

Ethanol 10.0%

Propylene glycol 5.0%

Phosphate buffer (0.05 M, pH 7.0): 100.0 mL

The ketorolac is dissolved in the phosphate buffer and pH of the solution is readjusted to 7.0 if necessary.

The solution is placed in an administrator designed to spray a specific volume to deliver therapeutically effective concentrations of ketorolac to the sublingual mucosa under the tongue.

### Example 2

Ketorolac tromethamine 10.0% (ketorolac acid equivalent)

Ethanol 10.0%

Propylene glycol 5.0%

Phosphate buffer (0.05 M, pH 7.0): 100.0 mL

The ketorolac is dissolved in the phosphate buffer and pH of the solution is readjusted to 7.0 if necessary.

The solution is placed in an administrator designed to spray a specific volume to deliver therapeutically effective concentrations of ketorolac to the sublingual mucosa under the tongue.

### Example 3

Ketorolac acid 10.0%

Ethanol 10.0%

Hydroxypropylmethyl cellulose 1.0%

Propylene glycol 5.0%

Phosphate buffer (0.05 M, pH 7.0): 100.0 mL

Approximately 70 mL of the phosphate buffer is heated to 80° C., and the hydroxypropylmethyl cellulose is dispersed in it with stirring. The ketorolac is dissolved in 30 mL of the phosphate buffer at 80° C., and the solution is mixed with the hydroxypropylmethyl cellulose dispersion. The resultant mixture is allowed to stand at room temperature for 3 hours.

The gel liquid formulation is placed in an administrator designed to spray a specific volume to deliver therapeutically effective concentrations of ketorolac to the sublingual mucosa under the tongue.

### Example 4

### An in vivo Absorbtion of Ketorolac Spray from the Sublingual Cavity

Experimental Technique

These experiments determine the bioavailability of ketorolac after transmucosal administration to the sublingual tissue in rabbit and compare it to that after intravenous administration.

The transmucosal absorption of ketorolac was measured using an in vivo technique in rabbits. Following introduction of lidocaine local anesthesia, a catheter was placed in the marginal ear artery of the rabbit for blood sample collection. For intravenous administration, a catheter was placed in the marginal ear vein of the rabbit and ketorolac aqueous solution (0.5 mg/kg, 0.1 mL) was administered; a sterile drug solution was prepared by filtration (double 0.22 μm filters). A dose of 2 mg/kg ketorolac was injected into the marginal ear vein cannula followed by a 0.1 mL flush with 10% (v/v) heparin/normal saline solution to keep the cannula patent.

For sublingual spray administration, the ketorolac dose (3 mg/kg, 0.1 mL) of the ketorolac formulation was applied by spray to the sublingual mucosa of the rabbit.

Aliquot parts of 0.5 mL blood samples were collected at predetermined time points, collected into pre-heparinized tubes and immediately placed on ice. Plasma was separated by centrifugation at 3000 rpm for 10 min, placed in polypropylene tubes, and frozen at −20° C. until the time of analysis.

Bioavailability of sublingually administered drug was calculated by comparing the plasma drug concentration between sublingual and intravenous delivery routes and expressed as a percentage of the intravenous bioavailability.

Formulation of the Transmucosal Dosage Form

The formulation instilled into the sublingual area of the rabbits in this experiment had the following composition:

Ketorolac acid 10.0%

Ethanol 10.0%

Sodium EDTA 0.02%

Tween 80 0.1%

Propylene glycol 5.0%

Phosphate buffer (0.05 M, pH 7.0): 100.0 mL

The ketorolac is dissolved in the phosphate buffer and pH of the solution is readjusted to 7.0 if necessary. The solution was placed in an administrator to spray a ketorolac dose to the sublingual mucosa under the tongue.

Sample Preparation

Plasma samples (45 μl) were spiked with the tolmetin internal standard solution (5 μl) then vortexed for 1 minute. A liquid-liquid extraction was carried out using Acetonitrile (50 μl) then followed by a 1 minute vortex and 10 minute centrifuge on 10,000 rpm. After centrifugation the supernatant was collected into HPLC vials in preparation for analysis by Ultra Performance Liquid Chromatography Mass Spectroscopy (UPLC/MS) system.

Assay Method for Ketorolac

All analytical procedures were performed using a Waters Acquity® Ultra UPLC/MS. The separation was achieved through a (2.1×100) Acquity UPLC® BEH Shield RP18 1.7 μm column, using two mobile phases delivered at 0.35 ml/minute flow rate with gradient ranging from 80% of (0.05% Formic Acid) at 1^{st} minute to 90% Acetonitrile over the next two minutes which then goes back to 20% at the 6^{th} minute of the run. The mass spectrometer was operated in the positive electrospray ionization (ESI) mode. The total run time was set at 10 minute. Mass chromatograms and mass spectral data were acquired and processed by MassLynx software (Waters).

Results

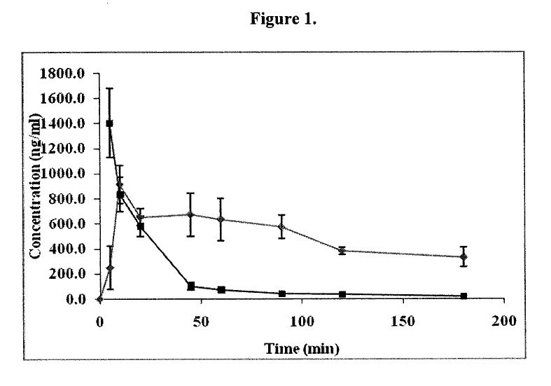

The pharmacokinetic profile of ketorolac was assessed in intact animals over 180 minutes. Mean ketorolac plasma concentration profiles versus time relationship that resulted after sublingual dosing to rabbits in comparison with intravenous administration were illustrated in FIG. 1.

Comparing the areas under the plasma concentration versus time curves (see FIG. 1) shows that ketorolac is rapidly and completely absorbed following transmucosal administration through the sublingual route, and the peak plasma concentration occurs at approximately 10 minutes following transmucosal administration. These results indicate that the sublingual spray formulations of ketorolac produce a rapid response with great bioavailability of the administered dose.

While this invention has been described as having a preferred embodiment, it is understood that the invention is not limited to the illustrated and described features. To the contrary, the invention is capable of further modifications, uses, and/or adaptations following the general principles of the invention and therefore includes such departures from the present disclosure as come within the known or customary practice in the art to which the invention pertains, and as may be applied to the central features set forth above, and which fall within the scope of the appended claims.

It would be obvious to those skilled in the art that modifications or variations may be made to the preferred embodiment described herein without departing from the novel teachings of the present invention. All such modifications and variations are intended to be incorporated herein and within the scope of the claims.

What is claimed is:

1. A pharmaceutical sublingual spray composition comprising ketorolac or a pharmaceutically acceptable salt thereof and a pharmaceutically acceptable carrier, wherein the ketorolac or pharmaceutically acceptable salt thereof is provided in a form suitable for sublingual administration.

2. A liquid spray formulation, comprising: (i) ketorolac or pharmaceutically acceptable salt or free acid thereof, (ii) buffered water; and (iii) a polar organic solvent, wherein the said polar organic solvent is present in an amount sufficient to enhance the solubility of the ketorolac free acid or salt thereof in the water.

3. The formulation of claim 2, wherein the ketorolac is present as the free acid or salt.

4. The formulation of claim 2, wherein the ketorolac is present as ketorolac tromethamine salt.

5. The formulation of claim 2, wherein the ketorolac is present as a ketorolac free acid.

6. The formulation of claim 2, wherein the ketorolac is present as a racemic mixture.

7. The formulation of claim 2, wherein the ketorolac is present as a pure ketorolac enantiomers.

8. The formulation of claim 2, wherein the ketorolac is present as a non-racemic mixtures of ketorolac enantiomers.

9. The formulation of claim 2, wherein the formulation is partially pressurized.

10. The formulation of claim 2, wherein the ketorolac or pharmaceutically acceptable salt or free acid thereof, is present at a concentration of 0.001%-80%.

11. The formulation of claim 2, wherein the ketorolac or pharmaceutically acceptable salt or free acid thereof, is present at a concentration of 0.01%-20% by weight.

12. The formulation of claim 2, wherein the polar organic solvent is an alcohol.

13. The formulation of claim 6, wherein the alcohol is selected from the group consisting of ethanol, propylene glycol, glycerol, polyethylene glycol and mixtures thereof.

14. The formulation of claim 2, wherein the polar organic solvent is present in an amount of 0-90% w/w.

15. The formulation of claim 2, wherein the formulation is buffered.

16. The formulation of claim 10, wherein the formulation is buffered with citrate or phosphate buffer.

17. The formulation of claim 2, wherein the formulation has pH of less than 10.

18. The formulation of claim 12, wherein the formulation has a pH of about 7.

19. The formulation of claim 2, wherein the carrier is aqueous solution, non-aqueous solution, or a combination of an aqueous solution and a non-aqueous solution thereof

20. The formulation of claim 2, wherein the carrier comprises solutions, gels, suspensions, liposomal dispersions, emulsions, microemulsions, nanoparticles and combinations thereof that can be sprayed via a conventional spray device.

21. A pharmaceutical composition of claim 20 wherein the composition further comprises at least one flavors agent, artificial coloring, sweetener, buffer, solvent, cosolvent, bioadhesive polymer, permeation enhancer, or buffering agent.

22. The composition of claim 21 wherein said bioadhesive agent is hydroxypropyl methylcellulose, a cellulose derivative, a natural gum, hyaluronates, chitosans, alginate, pectin, or combination thereof, present in from about 0.5 to about 15%, by weight.

23. The formulation of claim 2, further comprising a penetration enhancer.

24. The formulation of claim 23, wherein the penetration enhancer is a bile salt.

25. The formulation of claim 2, further comprising a muco-adherent.

26. The formulation of claim 25, wherein the muco-adherent is selected from the group consisting of chitosan, polyvinyl pyrrolidone, and gelatin.

27. The formulation of claim 2, wherein the formulation is suitable for sublingual administration.

28. A method of providing fast relief from the symptoms of pain, comprising administering to a subject in need thereof a pharmaceutically effective amount of ketorolac, by spraying the ketorolac onto the subject's sublingual mucosa.

29. The method of claim 28, wherein the ketorolac is in the form of ketorolac free acid or tromethamine salt, or any acceptable salt dissolved in an ethanolic solution.

30. A method of providing fast relief from the symptoms of pain comprising administering to a subject in need thereof the formulation of claim 2, by spraying the formulation onto the subject's sublingual mucosa.

31. A method for rapidly and reliably delivering therapeutically effective concentration of ketorolac to the systemic circulation of a patient for the treatment of pain using a spray administered sublingually under the tongue.
